(19) United States
(12) Patent Application Publication
(10) Pub. No.: US 2005/0145258 A1
(43) Pub. Date: Jul. 7, 2005

(54) Composition, Method and Device for Blood Supply Fluctuation Therapy

(76) Inventor: Yonghua Dong, Solon, OH (US)

Correspondence Address: Dr. Yonghua Dong, 32154 N. Roundhead Drive, Solon, OH, 44139, US

(21) Appl. No.: 10/905,455

(22) Filed: Jan. 5, 2005

(30) Foreign Application Priority Data
Jan. 5, 2004 (CN) 200410013838.0

(51) Int. Cl. A61B 19/00

(52) U.S. Cl. 128/898; 604/408

(57) ABSTRACT

The invention provides a method of blood supply fluctuation therapy (BSFT), which is useful in treating target tissues such as tumors. Also provided are a bio-absorbable BSFT agent such as blood soluble gaseous composition comprising CO₂ or O₂, and a device for storing and delivering gaseous BSFT agent, comprising a storing bag and a delivery system. The method is non-allergenic, non-nephrotoxic, safe, reliable, and cost-effective.

### BACKGROUND OF THE INVENTION

This application has foreign priority right of State Intelligent Property Office of the People's Republic of China, in which the application number is 20041 001 3838.0, filling date is Jan. 5, 2004.

The present invention is related to compositions, methods, and devices for blood supply fluctuation therapy (BSFT). The method is non-allergenic, non-nephrotoxic, safe, reliable, and cost-effective.

The importance of blood for life would never be overemphasized. Blood transports oxygen from the lungs to body tissue and carbon dioxide from body tissue to the lungs. It also conveys nourishment from digestion and hormones from glands throughout the body. Blood transports disease fighting substances to the tissue and waste to the kidneys.

All normal and diseased organs, even tumors, are dependent upon their blood supply for survival. Tumor is a common and often devastating disease. About 40% of the entire population in developed nations will be diagnosed with cancerous tumor during their lifetime, and half of these patients will die from the disease. As such, numerous efforts have been carried out to therapeutically damage a target tissue by reducing or stopping blood supply to the target tissue.

U.S. patent application 20030013759 to Das has disclosed a method of selectively reducing the blood supply to a neoplastic region, such as a tumor region, thereby selectively causing necrosis of the neoplastic tissue. In the method, blood vessels feeding the neoplastic region are selectively occluded by intra-arterial injection of polyunsaturated fatty acids. Moreover, U.S. patent application 20020018752 to Krall et al. teaches the application of a polymerizable composition for ablating diseased or undesired tissue by cutting off the blood supply to the tissue.

Advantageously, the present invention provides a new method, blood supply fluctuation therapy (BSFT), which is useful in treating target tissues such as tumors. The method is non-allergenic, non-nephrotoxic, safe, reliable, and cost-effective. The present invention also provides a bio-absorbable BSFT agent such as blood soluble gaseous composition comprising CO₂ or O₂, and a device for storing and delivering gaseous BSFT agent.

### BRIEF DESCRIPTION OF THE INVENTION

One feature of the present invention is to provide a new method of blood supply fluctuation therapy, which is useful in treating target tissues such as tumors.

Another feature of the present invention is to provide a bio-absorbable BSFT agent such as blood soluble gaseous composition comprising CO₂ or O₂.

Still another feature of the invention is to provide a device for storing and delivering gaseous BSFT agent.

### BRIEF DESCRIPTION OF THE DRAWINGS

FIG. 1 is a schematic elevational view of a system for storage and delivery of a BSFT agent in accordance with the present invention.

FIG. 2 is a schematic elevational view of a modified system for storage and delivery of a BSFT agent in accordance with the present invention.

FIG. 3 is a schematic elevational view of a system of storage and delivery for BSFT with carbon dioxide for the treatment of liver cancer.

FIG. 4 is a schematic elevational view of a modified system of storage and delivery for BSFT with carbon dioxide for the treatment of liver cancer.

FIG. 5 is a CT scan photo from a patient with liver cancer, showing that tumor is filled with carbon dioxide after injection of 50 ml CO₂ into the hepatic artery, only small gas is seen in normal liver tissue.

FIG. 6 is a CT scan photo from the same patient as that of FIG. 5, showing that about one half of tumor is filled with the gas 5 minutes injection. No gas is seen in normal liver tissue.

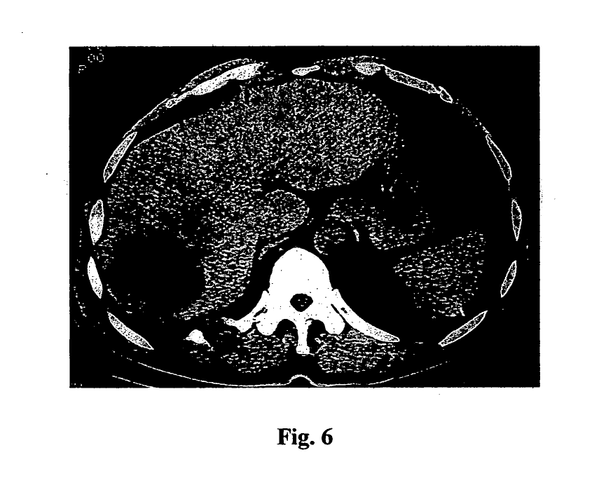

FIG. 7 is a CT scan photo from the same patient as that of FIG. 5, showing that less than 20% of tumor is filled with the gas 10 minutes after the injection.

FIG. 8 is a DSA imaging showing that CO₂ bubbles filled and accumulated in the immature tumor vasculature after 50 ml of CO₂ injection into hepatic artery in a patient with liver cancer, but no bubbles accumulated in the normal liver tissue.

FIG. 9 is an X-ray film showing that a liver tumor is fully filled with CO₂ after injection of a total of 500 ml CO₂ at the rate of 3 ml/s with 5 minutes interval each 50 ml injection, and the tumor looks like a balloon filled with gas.

FIG. 10 is an X-ray film from the same patient as that of FIG. 9, showing that, at the time of thirty minutes after injection of CO₂, about one half of the gas still remains in the tumor.

FIG. 11 is a CT scan photo from a patient with uterine fibroids, showing that “vapor lock” in tumor vasculature is produced by CO₂ 45 minutes after BSFT. No gas is seen in normal uterine tissue.

### DETAILED DESCRIPTION OF THE INVENTION

It is to be understood herein, that if a “range” or “group” or the like is mentioned with respect to a particular characteristic (e.g. temperature, ratio, time and the like) of the present invention, it relates to and explicitly incorporates herein each and every specific member and combination of sub-ranges or sub-groups therein whatsoever. Thus, any specified range or group is to be understood as a shorthand way of referring to each and every member of a range or group individually as well as each and every possible sub-ranges or sub-groups encompassed therein; and similarly with respect to any sub-ranges or sub-groups therein.

According to the present invention, when the blood supply system of a target tissue is filled with bio-absorbable agent, the blood supply to the target tissue will be relatively decreased (down-fluctuation); when the bio-absorbable agent present in the target tissue is bio-absorbed by its biological surroundings, the blood supply to the target tissue will be relatively increased (up-fluctuation). As used herein, the term “blood supply fluctuation” is defined as at least one cycle of the “down” and “up” fluctuations. Up-fluctuation and/or down-fluctuation will induce therapeutic effect on the target tissue, which constitutes the fundamental mechanism for the so-called blood supply fluctuation therapy (BSFT) of the present invention.

As one of its embodiments, the present invention provides a method of blood supply fluctuation therapy (BSFT method), which comprises:
(i) providing a target tissue in a mammal that is intended to be treated;
(ii) selecting one or more delivery sites in the blood supply system to the target tissue;
(iii) releasing a predetermined amount of bio-absorbable agent in the selected one or more delivery sites;
(iv) inducing down-fluctuation of the blood supply to the target tissue;
(v) inducing up-fluctuation of the blood supply to the target tissue when the bio-absorbable agent within the target tissue is and/or has been bio-absorbed; and
(vi) optionally repeating steps (ii) to (v) at least once.

There is no specific limitation to the mammal that is subject to the BSFT method, but preferably the mammal is a human. Target tissue may be normal or abnormal tissue such as tumorous tissue of any organ or part of a mammal such as human, including liver, spleen, pancreas, kidney, brain, spine, lungs, bone, heart, muscle, stomach, intestine, vessels, uterus, bladder, skin, gland, and the like. Preferably, the target tissue is located below the diaphragm, or in any other sites where reflux could not occur into the cerebral circulation.

Various components within the target tissue of a mammal such as human may be therapeutically damaged or altered by the BSFT method of the invention. Exemplary components within a target tissue include, but are not limited to, various cells and their cell membranes and intracellular organelles; extracellular medium such as fluid; abnormal vasculature; blood vessels such as arterial vessels, vascular vessels and venous vessels; blood vessel walls; and various blood cells and ingredients such as red blood cells, white blood cells, and platelets etc.

The therapeutic effects from the BSFT method of the invention include, for example, retarding the target tissue's growth, inhibition of the target tissue's growth, shrinkage of the target tissue's size, deactivation the function of the target tissue, blood vessel dysfunction, cells' necrosis, and cells' apoptosis etc.

The bio-absorbable agent used in the BSFT method of the invention may be in the form of solid, liquid, gas, or combination thereof. Exemplary BSFT agent may be traditional short-term and middle-term embolic agents that are made of solid materials and bio-absorbable, which make it possible to re-open the occluded vessels within a period of time. Examples of short-term embolic agent include blood clots or tissues from patients themselves, such as autologous blood clot and modified autologous blood clot. Examples of middle-term embolic agent include gelfoam (surgical gelatin sponge), and oxycel (oxidized cellulose) etc. Examples of heterogeneous combination of solid, liquid and gas include, but are not limited to, solid-in-gas colloid, liquid-in-gas colloid, solid-in-liquid suspension, and emulsion etc.

Preferably, the chemical, biochemical and biological properties of the BSFT agent should be such that (1) it can be substantially bio-absorbed within or through the target tissue region, for example, at least 10% by weight or volume, preferably at least 30%, more preferably at least 60%, even more preferably at least 80%, and most preferably at least 95% of the BSFT agent is absorbed within or through the target tissue; (2) it can be bio-absorbed within a period of time, for example, from one minutes to 5 days, preferably from 5 minutes to 3 days, more preferably from 10 minutes to 1 day, even more preferably from 30 minutes to 12 hours, and most preferably 45 minutes 6 hours; and (3) it is toxicologically acceptable if a small amount of the BSFT agent that has not been absorbed within the target tissue such as small gaseous bubbles, solid particles, or liquid droplets are displaced outside the target tissue. It should be understood that bio-absorption of the BSFT agent may include the disappearance of the BSFT agent as a single phase.

In some embodiments, the bio-absorbable BSFT agent used in the invention is gas or gas mixture, for example, at the temperature ranging from room temperature to a mammal's body temperature. As a skilled person in the art may appreciate, human or animal body temperature may be higher than the normal temperature such as 37°, for example, the temperature of a patient who suffers fever. Preferably, the gaseous BSFT agent is blood absorbable. By “blood absorbable”, it means that a BSFT agent may dissolve in blood aqueous phase, and/or bind to various blood components such as red blood cells e.g. hemoglobin, and white blood cells etc.

In various embodiments of the invention, the gaseous BSFT agent may be a composition comprising CO₂, a composition comprising O₂, or a composition comprising mixture of CO₂ and O₂. For example, the gaseous composition may be medical grade pure (>99.9%) CO₂ gas. Other gaseous species that can further be contained in the composition may be selected from the group consisting of ammonia, ozone, NO, H₂S, N₂, inert gases such as helium, neon, xenon, argon, and krypton, radioactive isotope thereof, and mixture thereof. The concentration of various gaseous components in the composition may be determined by a skilled artisan, in light of the established requirements that has been described above. For example, since CO₂ has blood solubility ˜20 times higher than O₂, when a mixture of CO₂ and O₂ is used as the BSFT composition, the volume ratio between CO₂ and O₂ may be adjusted so that desirable magnitude and frequency of the blood supply fluctuation may be obtained. For example, the volume ratio between CO₂ and O₂ may be in the range of from 99:1 to 1:99, preferably in the range of from 90:10 to 60:40.

To obtain desirable magnitude and frequency of the blood supply fluctuation, the amount and ingredients of the BSFT composition used for each cycle of up-down fluctuation may be determined by various factors such as blood flow, target organ physiology, target organ anatomy, target tissue size, target tissue structure, BSFT composition delivery site(s), blood vessel abundance, blood vessel distribution, blood vessel anatomy, and many other special factors such as involvement of brain blood barrier etc. Taking blood vessels as example, they are actually not uniform and generic. Blood vessels are strikingly heterogeneous and different from one organ to another organ and from normal organ to diseased organ.

Although, as described above, any tissue may be the target of the invention, in preferred embodiments, abnormal tissues such as those suffer from tumor (neoplasm); hemangioma, vascular malformation, infection, and splenomegaly etc. are subject to the BSFT therapy of the invention. In a specific embodiment, the gaseous BSFT composition comprising CO₂ is used to treat tumors such as liver cancer.

Typically, a BSFT composition comprising CO₂ can be absorbed and disappeared in less a minute in normal vasculature, but CO₂ can accumulate and stay in abnormal vasculature of e.g. liver tumorous tissue for hours, even for days. This significantly prolonged dwelling of BSFT composition comprising CO₂ in tumorous tissues is presumably due to immature characteristics of tumor vasculature such as long and entangled loops, irregular vessel diameters than normal value, and etc. It is believed that angiogenesis, or neovascularization, is responsible for these immature characteristics of tumor vasculature. After the pre-vascular stage of “carcinoma in situ”, some rapidly dividing tumor cells are known to acquire the ability to express genes encoding angiogenesis growth factors, which are diffused into the nearby tissues. When the angiogenic growth factors are binded to specific receptors located on the endothelial cells (EC) of nearby preexisting blood vessels, the cells are activated to produce enzymes that are able to dissolve tiny holes in the sheath-like covering (basement membrane) surrounding all existing blood vessels. The endothelial cells then begin to divide or proliferate and migrate out through the dissolved tiny holes towards the tumorous tissue. Adhesion molecules or integrins function as grappling hooks to help the sprouting new blood vessel sprout forward. In the meanwhile, additional enzymes such as matrix metalloproteinases or MMP are produced to dissolve the tissue in front of the sprouting vessel tip in order to accommodate it. As the vessel extends, the tissue is remolded around the vessel. Sprouting endothelial cells roll up to form blood vessel tubes, which may then connect to each other, forming blood vessel loops that can circulate blood. Of course, a skilled person in the art can understand that other characteristics of tumor vasculature may also play a role in the BSFT method of the invention, such as widened lumens, aneursymal dilatations, fewer associated smooth muscle cells and pericytes, irregular blood flow, regions of stasis, and high permeability etc.

Typically, for a gaseous BSFT composition comprising CO₂, a composition comprising O₂, or a composition comprising mixture of CO₂ and O₂, the volume may be from 5 ml to 10000 ml, preferably from 20 ml to 6000 ml, more preferably from 50 ml to 3000 ml, and most preferably from 100 ml to 2000 ml. If a large target tissue is to be embolized, performing the BSFT procedure in separate stages is advisable in order to prevent widespread tissue necrosis, to allow time for normal tissues recover, and to permit early detection of complications.

In preferred embodiments, overdose or under-dose of the BSFT composition is limited to a minimum level by precisely monitoring and evaluating the therapeutically procedure. For example, gaseous BSFT composition such as CO₂ has been shown to be excellent in displacing blood and creating a void in vessels, which can be visualized with Digital Subtraction Angiography (DSA), Computer Tomography (CT), MRI, and Ultrasound (US) etc.

The blood supply fluctuation therapy according to the present invention may employ many known techniques of vascular interventional procedure, for example, vascular embolotherapy such as transcatheter vascular embolotherapy. For example, the BSFT bio-absorbable agent may be used as a vascular embolic agent to occlude blood flow of a given vessel for down-fluctuation, and then be bio-absorbed to partially or completely resume the blood flow for up-fluctuation.

According to one embodiment of the invention, at one or more delivery sites, a gaseous BSFT composition is selectively delivered into the vessels of the targeted tissues or organs. The delivery of BSFT composition may be accomplished by intravascular route such as intra-arterial route, intravenous route, intracapililary route, intracardial route, etc. or combination thereof. For example, if catheters are used in the BSFT method of the invention, they should be placed as selectively as possible in order to avoid embolization of normal tissues. Preferably, none or minimum of emboli is refluxed from the catheterized vessels such as arteries.

In the case of intravascular administration, it is preferable to resort to introducing a catheter into the blood vessel of the target tissue that is to be treated. Catheters that can be used for this purpose are those that are commonly used in the technique. The nature of the material constituting the catheter is not critical in itself; nevertheless, a flexible material such as silastene is preferred. It may be implanted in the blood vessel as is commonly done, for example, after local anesthesia and incision. For greater convenience, the catheter can be held in place by a suture, after which the incision may or may not be closed up.

Selectivity is critically important for the BSFT therapy of the invention and special attention should be paid to the issue. For instance, to treat a patient with liver cancer, precise evaluation of the number and site of target tissue before the BSFT therapy is essential for the accurate planning of surgery and the avoidance of unnecessary damage on normal tissue, especially in patients with liver dysfunction.

Down-fluctuation of blood supply may be accomplished via different modes. For example, the gaseous bio-absorbable composition of the present invention may form a local vapor lock area by displacing the blood within a segment of the vascular lumen (e.g. embolism) within the target tissue, which can effectively block blood supply and nutrition into the target tissue, such as tumor etc. The term embolism indicates an obliteration of a blood vessel by a clot or a foreign body moved by the blood up to the place where the clearance of the blood vessel is insufficient to permit its passage. For another example, the gaseous bio-absorbable composition may generate some big “bubbles” that move slower than flowing blood or even arrest inside blood vessels, lowering the blood supply rate available to the target tissue. For still another example, the gaseous bio-absorbable composition may generate smaller “bubbles” that, although move as fast as the flowing blood inside the blood vessels, at least transiently limit the blood supply rate available to the target tissue because some blood is replaced by the bubbles. It should be understood that the invention also includes various combinations of the down-fluctuation modes that have been described above.

Therapeutic effects of blood supply down-fluctuation on target tissues may be, for example, ischemia, hypoxia, cell necrosis and/or apoptosis induced from ischemia, and other effects that are known to a skilled person in the art lschemia is defined in the present invention as a condition, in which target tissues suffer from inadequate oxygen and/or nutrient. An exemplary ischemic damage is to the lipid portion of cell membranes through lipid peroxidation and phospholipase activity. For example, blood supply down-fluctuation of the invention may reduce or even stop the nutrition which is necessary to keep the target cells, tissues and organ functioning, living and growing etc.

Up-fluctuation of blood supply may also be accomplished via different modes. For example, a local vapor lock in target tissue may be downsized via gradual bio-absorption by various components within and around the target tissue such as blood. A local vapor lock may be downsized to smaller vapor lock, to big bubbles that move slower than flowing blood, or even small bubbles that move up to the normal speed of flowing blood. By the same token, big bubbles may be downsized to small bubbles and flow out from the target tissue; and small bubbles may be further downsized, or flow out from the target tissue, or completely be bio-absorbed such as dissolved within the target tissue. It should be understood that the invention also includes various combinations of the up-fluctuation modes that have been described above.

Therapeutic effects of blood supply up-fluctuation on target tissues may be, for example, reperfusion injury, blood vessel damaging, induced cell necrosis and/or apoptosis, blood vessel dilation, improved O₂ and nutrition availability, and other effects that are known to a skilled person in the art. In preferred embodiments of the invention, reperfusion injury refers to the tissue damage inflicted when blood supply is completely or partially restored after a down-fluctuation (e.g. ischemia) period of more than about ten minutes.

Sometimes, up-fluctuation of blood supply may be more damaging than down-fluctuation (e.g. ischemia) because ischemia sets the stage for oxygen to generate free-radicals rather than contribute to cellular energy production. It is believed that within a certain period of time such as several minutes without blood supply, cells of target tissues lack the energy source such as ATP, due to ATP is broken-down to xanthine, and cells attempt to produce ATP by anaerobic glycolysis. Xanthine and oxygen may be converted to superoxide & uric acid by endothelial enzyme xanthine oxidase.

In some embodiment of the invention, reperfusion damaging may take place on endothelial cells as well as platelets, leucocytes and other cells in the blood stream. For example, eicosanoids (leukotrienes & prostaglandins) and associated oxygen free-radicals may damage the endothelium, not only increasing edema (tissue swelling due to “leakiness”), but also causing endothelial protrusions (“blebs”) which can block capillaries. These effects quickly become pronounced enough in reperfusion to block capillaries entirely. Free-radical and other membrane damage can loosen or dislodge atherosclerotic plaque causing emboli upon reperfusion.

Of course, other factors may play a role on the therapeutic effect of the BSFT method of the invention. For example, the effects of embolization on any organ are specific to that organ and the clinical status of the patient.

Any suitable device or apparatus that can store the bio-absorbable BSFT material and deliver it into targeted tissue may be used in the invention. For example, gaseous BSFT compositions such as CO₂, O₂, or mixture of CO₂ and O₂ can be stored in a cylinder or a flexible bag. A syringe may be connected to the cylinder or a flexible bag, and then filled with the gaseous BSFT composition. The syringe may then be disconnected from the cylinder or a flexible bag and connected to a catheter or tube set. If desired, the syringe may be disconnected from the catheter and refilled with the gaseous BSFT composition from the cylinder or a flexible bag. This procedure may be repeated for many times. An operator should be careful and not to introduce undesired material such as air into the system, for example, at every disconnection.

Preferably, multiple submicron filters are used with gas cylinder to avoid contamination with water, rust, and particulate material.

In an embodiment of the invention, one or more cylinders containing gaseous BSFT composition may be attached directly to a stopcock with a syringe attached at on port and the catheter to the patient attached to the other port. When the syringe is to be filled, the stopcock is opened to the syringe and the cylinder pressure will force the gaseous BSFT composition into the syringe. For injection into the patient, the stopcock is closed to the cylinder and the syringe plunger is advanced forward pushing the gaseous BSFT composition into the catheter and, subsequently, into the patient.

According to a specific embodiment, the invention provides a device for storing and delivering gaseous BSFT composition such as vascular embolic agent to the target tissue of a patient. The device comprises a storing bag and a delivery system. The storing bag comprises an inlet tubular member, a storing chamber (bag), and an outlet tubular member. One end of the inlet tubular member connects with one side of the storing bag, and another end of the inlet tubular member connects to the gaseous BSFT composition resource(s). One end of the outlet tubular member connects with another side of the storing bag and another end of the outlet tubular member connects with the delivery system. The delivery system may comprises a first check valve, “T” shape three-way connecting tubular member, a second check valve, a third check valve, and a three-way stopcock etc.

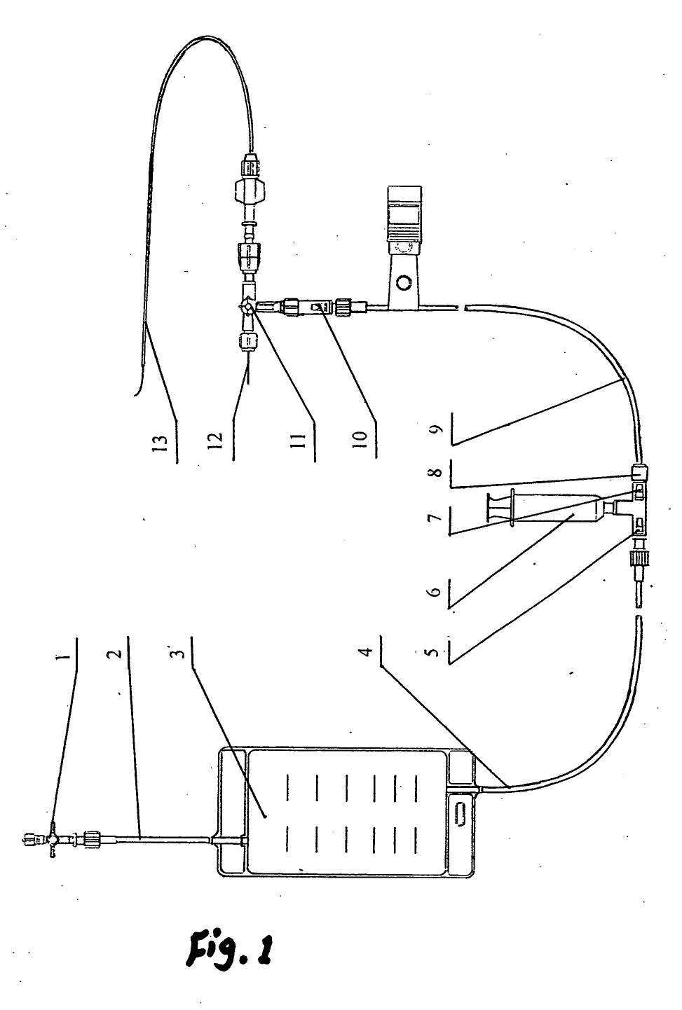

Refereeing now to FIGS. 1-4, the figures are example embodiments showing systems for storing and delivering gaseous BSFT composition such as vascular embolic agent comprising a storing bag 3 and a delivery system. The storing bag 3 has an inlet tubular member 2 and an outlet tubular member 4. One end of the inlet tubular member connects to one side of the bag, and another end of the inlet tubular member 2 connects to the gaseous BSFT composition resource. One end of the outlet tubular member connects to another side of the storing bag and another end of the outlet tubular member connects to the delivery system. The delivery system comprises a first check valve 5, a “T” shape three-way connecting tubular member 8, a second check valve 7, a connecting tubular member 9, a third check valve 10, and a three-way stopcock 11. Two ends of the straight arm of the “T” shape three-way connecting tubular member 8 connect to the first check valve 5 and the second check valve 7. The first check valve connects to the outlet tubular member 4. The second check valve connects to the connecting tubular member 9. The middle port of the “T” shape three-way connecting tubular member 8 connects to a pump or syringe 6. The downstream outlet of the connecting tubular member 9 connects to the third check valve 10. The middle port of the stopcock 11 connects to the outlet of the third check valve 10. The outlet of the straight arm of the three-way stopcock 11 connects to a delivery catheter 13. A guide wire 12 passes through the straight arm of the three-way stopcock 11 into the catheter 13. The delivery catheter 13 may be a conventional single lumen catheter or dual lumen occlusive balloon catheter.

In a specific embodiment, the first check valve 5 and the second check valve 7 connect to the two ends of the straight arm of the “T” shape connecting tubular member 8 respectively.

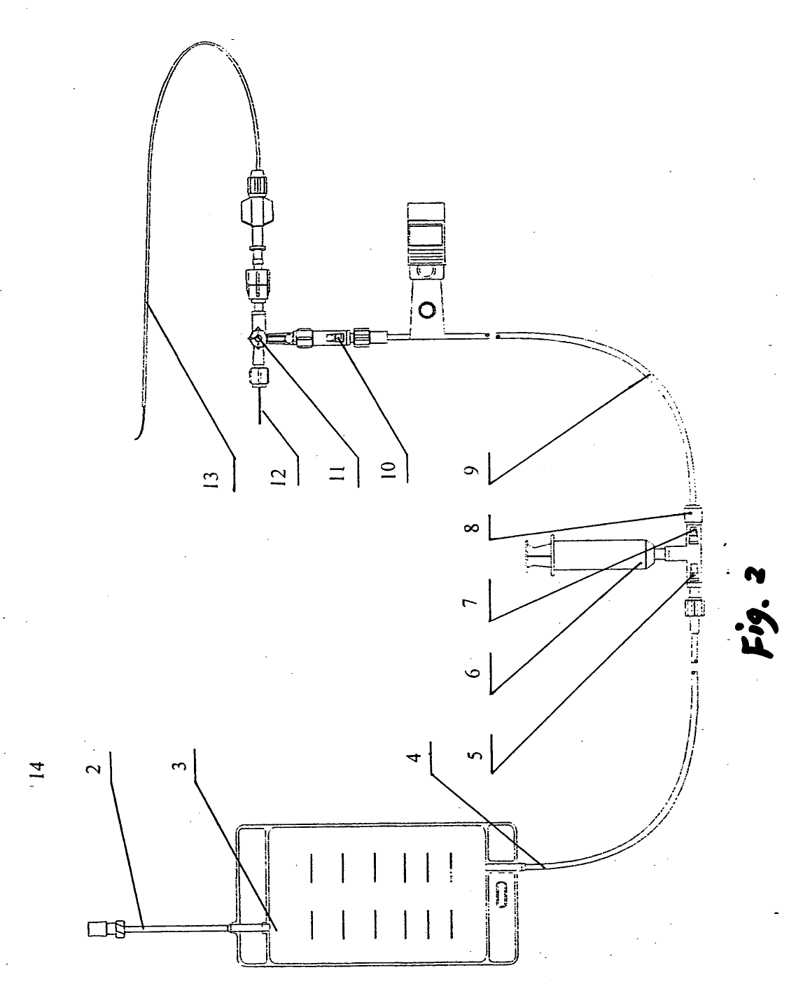

In a specific embodiment, the inlet of the tubular member 2 has a stopcock 1 or a valve 14, as shown in FIG. 2. When the inlet of the storage bag connects to resource of gaseous embolic agent, gaseous embolic agent enters into the storage bag directly, and flushes and cleans the air within the storage bag and delivery device.

According to a specific embodiment, the device for storing and delivering gaseous BSFT composition such as vascular embolic agent to the target tissue of a patient may be controlled automatically, such as under the control of a computer system.

Numerous benefits can be obtained from using the method of the present invention. For example, the blood supply fluctuation therapy is non-allergenic, non-nephrotoxic, safe, reliable, and cost-effective.

In an embodiment of the invention, gaseous BSFT composition comprising CO₂ or O₂ or mixture of CO₂ and O₂ may also be used to monitor or visualize the blood vessels in target tissue. Fro example, CO₂ displaces blood and is imaged by the differential density of the gas compared to the surrounding tissues.

Particular advantages over prior art are provided by the BSFT method of the invention, in which gaseous BSFT composition comprising CO₂ or O₂ or mixture of CO₂ and O₂ is used. The gaseous BSFT composition is safe, cheap and effective in blocking blood supply to the target tissue and inducing blood supply fluctuation. For example, carbon dioxide can be absorbed rapidly by blood and exhaled from lungs. Carbon dioxide does not cause embolization in normal vessels, and therefore no harm will be produced to normal tissue and organ if it is delivered into normal vessels by accident. Carbon dioxide has no nephrotoxicity and hepatotoxicity, and it can be used in the patients with renal and hepatic function impairment. Due to carbon dioxide is also a material produced in human body, it will not induce allergenic response and can be used in patients with allergic diseases.

Moreover, gaseous composition comprising CO₂ or O₂ or mixture of CO₂ and O₂ can selectively accumulate in tumor vessels and form “vapor lock”, blocking blood supply to tumor that make tumor starve and die, among other damages. Due to the gaseous composition produces non-blood embolization, inflammation response such as swelling, pain, fever and abscess will be alleviated or removed, and patients' toleration capability to the BSFT including embolization will be improved.

In contrast, in interventional treatment of e.g. liver cancer, due to the abnormal shunting between hepatic artery and portal vein or between hepatic artery and hepatic vein caused by the invasion of cancer, during the delivering of e.g. iodized oil for embolization, iodized oil can pass the abnormal shunting entering into portal vein to cause ischemia of normal liver, or even into pulmonary artery to cause infarct of normal lung tissue. These phenomena can cause unnecessary severe damage to normal tissue and body, while blood supply to tumor is not satisfactorily occluded, and consequently the therapeutic efficacy is limited.

The storage and delivery device of the gaseous BSFT composition is also easy to build and reliable in operation. Carbon dioxide and oxygen are very easy to produce, or to obtain commercially, and they need no expensive equipment for storage. In some embodiments, the delivery device is a closed system, and the storage and delivery of BSFT composition such as gaseous embolic agent is simple and able to save operation time. The device is also safe and reliable for interventional procedure. For example, the third check valve connects to middle port of the third three-way stopcock, and the two ends of the straight arm on the third three-way stopcock are used for connecting to syringe and catheter respectively.

In an BSFT embodiment, flush saline or BSFT agent can be injected with syringe into vessels through catheter, and guide wire can be inserted or exchanged through the straight arm of the third three-way stopcock and catheter without disconnecting catheter. This solves the problem existing in prior intervention procedure that catheter must be disconnected if guide wire insertion and exchange are required. When a catheter is disconnected, the catheter lumen is open to air, and the catheter has to be flushed again before re-connection. Therefore, the present invention provides a much simpler and time-saving procedure, and also improves the safety and reliability of BSFT treatment.

Without further elaboration, it is believed that one skilled in the art can, using the description herein, utilize the present invention to its fullest extent. The following examples are included to provide additional guidance to those skilled in the art in practicing the claimed invention. The examples provided are merely representative of the work that contributes to the teaching of the present application. Accordingly, these examples are not intended to limit the invention, as defined in the appended claims, in any manner.

### EXAMPLES

### Example 1

### Blood Supply Fluctuation Therapy with Gaseous CO₂ for the Treatment of Liver Cancer

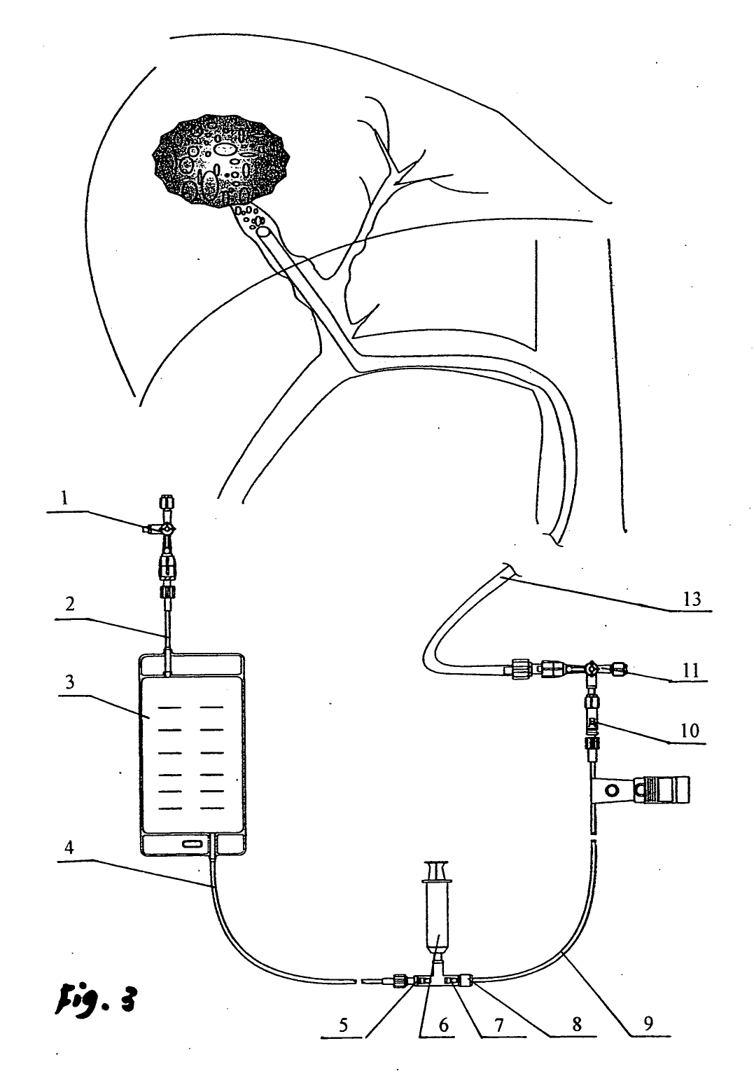

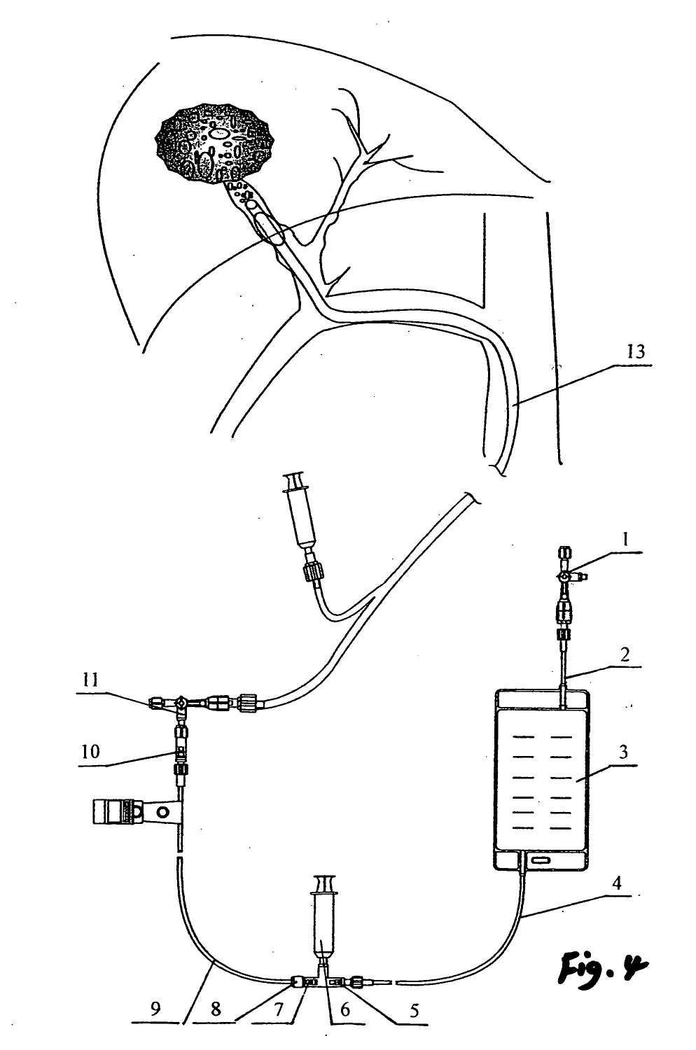

Preparation of gaseous CO₂: a sterile storing bag 3 (note: all the number here is referring to FIGS. 3 and 4) and delivery device was obtained. The inlet tube member 2 was connected to gas resource of gaseous CO₂. 500 ml of the gaseous CO₂ was introduced into storing bag for cleaning the air within the storing bag and delivery system. T Stopcock 1 in inlet tube member 2 was turned off to stop the gas into storing bag temporally. The gaseous CO₂ in storing bag 3 was aspirated into syringe 6, then impelled by the syringe 6. Due to the function of check valve, during the aspirating of syringe, the first check valve 5 was open; meanwhile the second and third check valves were closed automatically. The gas in the storing bag was aspirated into syringe 6. During the impelling of syringe, the first check valve was closed and the second and third check valves were open automatically. The gaseous CO₂ in the syringe is impelled out of the delivery device through the outlet of the third three-way stopcock. Repeating syringe aspirating and impelling until all gas in the storing bag was emptied, and the air within storing bag and delivery device was cleaned. After that, the stopcock on the inlet tubular member was turned on again to introduce the gaseous CO₂ from the gas resource into storing bag 3 to fill the storing bag (2000 ml), then the stopcock 1 was turned off to stop gaseous CO₂ introduction. The gaseous CO₂ and delivery system was ready for use.

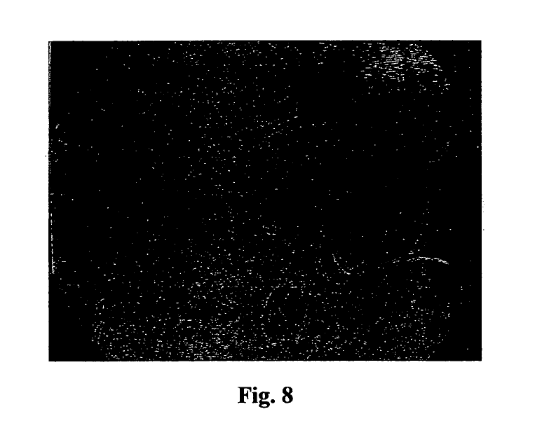

Selective catheterization and diagnostic Digital Subtraction Angiogrpahy (DSA) with Gaseous CO₂: under monitoring of X-ray fluoroscope and using interventional technique of selective catheterization as shown in FIG. 3, a delivery catheter 13 was inserted into the hepatic artery of a patient with liver cancer. The delivery catheter 13 was connected to the outlet of the third three-way stopcock of the delivery system, and the air within the catheter 13 was removed. Using the gaseous CO₂ as contrast medium, 50 ml gaseous CO₂ at rate of 10 ml/second was injected by syringe 6 into the hepatic artery to perform a diagnostic DSA. The diagnostic DSA was used to identify the location, size of tumor, arteries supplying blood to liver cancer, blood flow within tumor tissue and time gaseous CO₂ staying in tumor vessels. All those information was necessary to doctor to select an optimal protocol of Blood Supply Fluctuation Therapy (BSFT). Then, the angiographic catheter was advanced into the artery supplying blood flow to the liver cancer. FIG. 8 was a DSA imaging showing that CO₂ bubbles filled and accumulated in the immature tumor vasculature after 50 ml of CO₂ injection into hepatic artery in a patient with liver cancer, but no bubbles accumulated in the normal liver tissue.

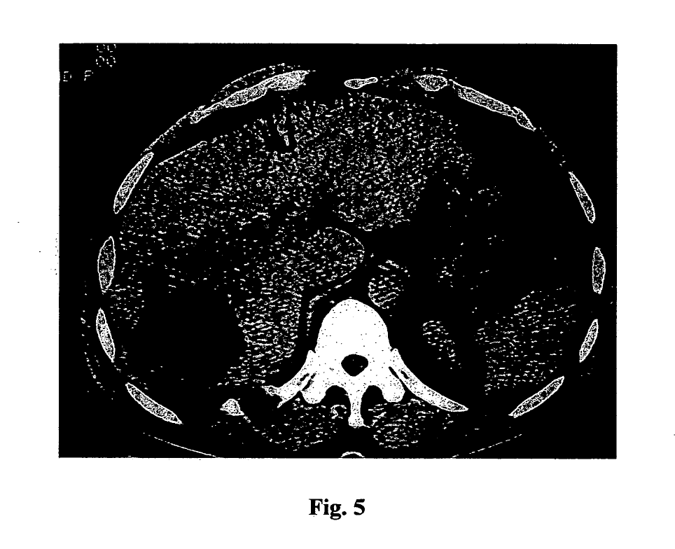

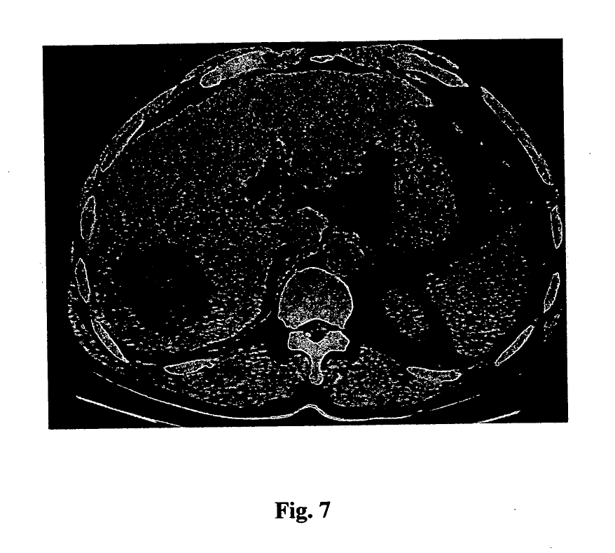

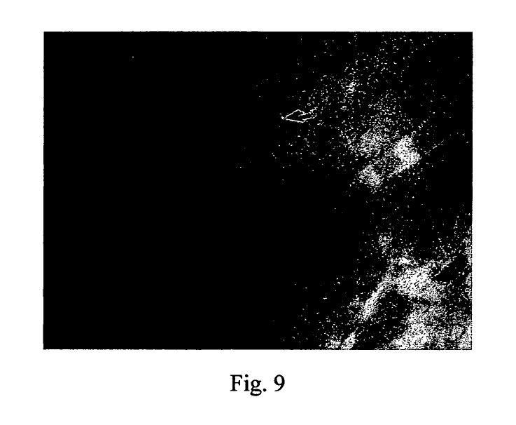

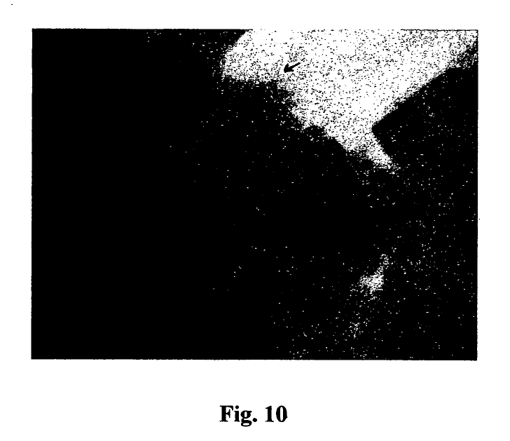

Blood Supply Fluctuation Therapy (BSFT) with Gaseous CO₂: gaseous CO₂ was injected slowly by the delivery syringe 6 through the catheter 13 into the tumor vasculature. Using CT scan, It was found that just after the first injection of 50 ml CO₂ into hepatic artery, the tumor vasculature was filled with the gas, and 5 minutes after the first injection, one half of the tumor vasculature still was filled with the gas (FIG. 5, 6), and less than 20% of the tumor was filed with CO₂ bubbles (FIG. 7) 10 minutes after the injection. Then, the second 50 ml injection was followed and tumor vasculature was filled with the gas again. The gas displaces the blood flow within the tumor vessels partially or entirely depending on the injection rate and lasting time. With increasing of the gaseous mixture, the blood supply to the liver cancer was decreased, even stopped. Because tumor vasculature was immature, “vapor lock” in tumor vasculature was produced by the gaseous CO₂. With decreasing and stopping of the blood flow of tumor vasculature, the gaseous CO₂ injected through delivery catheter might overflow into the arteries supplying normal liver tissue that could be detected and monitored by X-ray fluoroscopy, DSA, CT or ultrasound imaging. Because the gaseous CO₂ was absorbable in blood and could be flushed away in normal vasculature within one minute, therefore the gaseous CO₂ overflowing into normal vasculature caused no damage in normal liver tissue. At this time, gaseous CO₂ delivery was stopped. Due to vapor lock, there was no blood flow in tumor vasculature and no nutrition and oxygen was delivery to tumor cells, which made it under starvation and hypoxia. Meanwhile also due to the vapor lock, area of the CO₂ bubbles contacting with blood was getting much smaller, which limits CO₂ absorbability and prolongs CO₂ gas staying within tumor vasculature. We have found that a liver tumor with size of 7 cm in diameter could be fully filled with CO₂ after injection of a total of 500 ml CO₂ at the rate of 3-5 ml/s with 3-5 minutes interval each 50 ml injection. The patients receiving the injection of CO₂ only felt minor discomfort on liver area. Under X-ray fluoroscopy, the tumor looked like a balloon filled with gas (FIG. 9). Thirty minutes after injection of CO₂ about half of the gas still remained in the tumor (FIG. 10).

To achieve tumor apoptosis and necrosis, the intermittent injection of CO₂ can last hours to weeks according to tumor size, types and response to the blood supply fluctuation therapy. For the patients with advanced and large tumor a long term protocol is needed. The delivery catheter is remained in place and the patients is moved back to ward from operation room. A bedside portable ultrasound imaging unit is used to monitor the gas injection, distribution and gas filling in tumor vasculature. CO₂ delivery can be operated manually or by a computer controlled programmed injector that can deliver the gas into patients at different rate, pressure, amount and interval time, et al as needed. With the blood supply fluctuation, tumor suffers from both embolization and reperfusion injury.

If blood flow supplying tumor is fast, a balloon occlusion catheter can be used as shown on FIG. 4. During the injection of the gas the balloon is inflated to decrease or occlude the local blood flow temporary that benefits the gas accumulation within tumor vasculature. During the reperfusion, the balloon is flatted to increase the blood flow into tumor vasculature that induces much more reperfusion injury. The blood supply fluctuation can be terminated when tumor vasculature is destroyed or vapor locked and no more blood supply into the tumor. Tumor apoptosis and necrosis can be achieved by the treatment.

### Example 2

### Blood Supply Fluctuation Therapy with Gaseous Mixture of CO₂ and Nitric Oxide (NO) for the Treatment of Uterine Fibroids Diseases

Preparation of gaseous mixture of CO₂ and Nitric Oxide: the preparation procedure was same as that of gaseous CO₂ in example 1, except the gaseous material was not CO₂, but the gaseous mixture of CO₂ and Nitric Oxide (NO). The concentration of NO was present in an amount effective to prevent vasospasm and ischemia, 40 ppm here.

Selective catheterization and diagnostic Digital Subtraction Angiogrpahy (DSA) with Gaseous Mixture of CO₂ and Nitric Oxide (NO): under monitoring of X-ray fluoroscope and using interventional technique of selective catheterization, a delivery catheter was inserted into the internal iliac artery in the patients with uterine fibroids. An angiographic catheter was connected to the outlet of the third three-way stopcock of the delivery system, and the air was removed within the catheter. Using the gaseous mixture as contrast medium, 30 ml of the gaseous mixture was injected by a syringe at rate of 10 ml/second into the internal iliac artery to perform a diagnostic DSA. The diagnostic DSA was used to identify the location, size of tumor, arteries supplying blood to uterine fibroids, blood flow within tumor tissue and time gaseous mixture staying in tumor vasculature. All those information was necessary to doctor to select an optimal protocol of Blood Supply Fluctuation Therapy (BSFT). Then, the catheter was advanced into the vessels supplying blood flow to the uterine fibroids. Because uterine artery was very sensitive to catheter and guide-wire manipulation and injection, vasospasm was very common during the interventional procedure. The NO in the gaseous mixture was useful to prevent vasospasm.

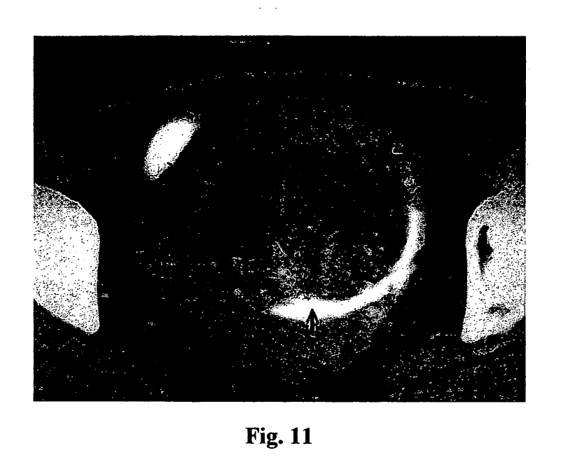

Blood Supply Fluctuation Therapy (BSFT) with Gaseous Mixture of CO₂ and Nitric Oxide (NO): gaseous mixture of CO₂ and Nitric Oxide (NO) was injected slowly and intermittently by the delivery syringe through the delivery catheter into tumor vasculature at the rate of 1 ml/s and with 5 minutes interval each 20 ml injection. The gas displaced the blood flow within the tumor vessels partially or entirely depending on the injection rate and lasting time. With increasing of the gaseous mixture, the blood supply to the uterine fibroids was decreased, even stopped. Because tumor vasculature was immature, “vapor lock” in tumor vasculature was produced by the gaseous mixture (FIG. 11). With the decreasing and stopping of the blood flow of tumor vasculature, the gaseous mixture injected through delivery catheter might overflows into the arteries supplying normal uterine wall that could be detected and monitored by X-ray fluoroscopy, DSA, CT or ultrasound imaging. Because the gaseous mixture was absorbable in blood and could be flushed away in normal vasculature within one minute, therefore the gaseous mixture overflowing into normal vasculature caused no damage in normal uterine tissue, and also NO in the gaseous mixture was useful to prevent vasospasm. At this time, gaseous mixture delivery was stopped. Due to vapor lock, there was no blood flow in tumor vasculature and no nutrition and oxygen are delivery to tumor cells, which made them under starvation and hypoxia. Meanwhile also due to the vapor lock, area of the gaseous bubbles contacting with blood was getting much smaller, which limited gaseous mixture absorbability and prolongs gaseous mixture staying within tumor vasculature. We have found that uterine fibroids with size of 5 cm in diameter could be fully filled with the gaseous mixture after injection of a total of 100 ml the gaseous mixture. Vasospasm was prevented effectively by the NO within the gaseous mixture, and the patients receiving the injection of gaseous mixture only felt minor discomfort.

### Example 3

### Blood Supply Fluctuation Therapy with Gaseous Mixture of CO₂ and Nitric Oxide (NO) and Gaseous Mixture of CO₂ and Oxygen for the Treatment of Lung Cancer

Preparation of gaseous mixture of CO₂ and Nitric Oxide and gaseous mixture of CO₂ and Oxygen: the preparation procedure will be same as the preparation of gaseous mixture in Example 2, except two kinds of gaseous materials are needed, which are gaseous mixture of CO₂ and Nitric Oxide (NO), the concentration of NO is present in an amount effective to produce enough Nitrous Dioxide to destroy tumor vasculature and kill tumor cells, prevent vasospasm and ischemia, preferably between 40 and 400 ppm; gaseous mixture of CO₂ and Oxygen, the concentration of Oxygen is present in an amount effective to produce enough Nitrous Dioxide to destroy tumor vasculature and kill tumor cells, preferably between 2% and 20% ppm.

Selective catheterization and diagnostic Digital Subtraction Angiogrpahy (DSA) with Gaseous Mixture of CO₂ and Nitric Oxide (NO): under monitoring of X-ray fluoroscope and using interventional technique of selective catheterization, a catheter will be inserted into the bronchial artery or intercostals artery in the patients with lung cancer. The delivery catheter will be connected to the outlet of the third three-way stopcock of the delivery system, and the air within the catheter should be removed. Using the gaseous mixture as contrast medium, 10-15 ml gaseous mixture at rate of 5-8 ml/second will be injected by syringe into the bronchia or intercostal artery to perform a diagnostic DSA. The diagnostic DSA can be used to identify the location, size of tumor, arteries supplying blood to the lung cancer, blood flow within tumor tissue and time gaseous mixture staying in tumor vasculature. All those information is necessary to doctor to select an optimal protocol of Blood Supply Fluctuation Therapy (BSFT). Then, the catheter will be advanced into the vessels supplying blood flow to the lung cancer. Because bronchial and intercostal arteries are very sensitive to catheter and guidwire's manipulation and injection, vasospasm is very common during the interventional procedure. NO in the gaseous mixture will be useful to prevent vasospasm.

Blood Supply Fluctuation Therapy (BSFT) with Gaseous Mixture of CO₂ and Nitric Oxide (NO): gaseous mixture of CO₂ and Nitric Oxide (NO) will be injected slowly and intermittently by the delivery syringe through the catheter into tumor vasculature at the rate of 1-2 ml/s and with 3-5 minutes interval each 10 ml injection. The gas displaces the blood flow within the tumor vessels partially or entirely depending on the injection rate and lasting time. With increasing of the gaseous mixture, the blood supply to the lung cancer will be decreased, even stopped. Because tumor vasculature is immature, “vapor lock” in tumor vasculature should be produced by the gaseous mixture. With the decreasing and stopping of the blood flow of tumor vasculature, the gaseous mixture injected through delivery catheter may overflows into the arteries supplying normal lung tissue that can be detected and monitored by X-ray fluoroscopy, DSA or CT. Because the gaseous mixture is absorbable in blood and can be flushed away in normal vasculature within one minute, therefore the gaseous mixture overflowing into normal vasculature could cause no damage in normal lung tissue, and also NO in the gaseous mixture is useful to prevent vasospasm. At this time, gaseous mixture delivery will be stopped. Due to vapor lock, there will be no blood flow in tumor vasculature and no nutrition and oxygen are delivery to tumor cells, which makes them under starvation and hypoxia. Meanwhile also due to the vapor lock, area of the gas bubbles contacting with blood is getting much smaller, which limits gas absorbability and prolongs gas staying within tumor vasculature. Vasospasm will be prevented effectively by the NO within the gaseous mixture, and the patients receiving the injection of gaseous mixture will feel very minor discomfort.

Blood Supply Fluctuation Therapy (BSFT) with Gaseous Mixture of CO₂ and Oxygen/Ozone: After the first Blood Supply Fluctuation Therapy (BSFT) with Gaseous Mixture of CO₂ and Nitric Oxide, Blood Supply Fluctuation Therapy (BSFT) with Gaseous Mixture of CO₂ and Oxygen will be followed with same procedure as above. Except inducing of embolization and reperfusion injury in tumor, toxicant nitrous dioxide will be produced within tumor vasculature. The toxicant nitrous dioxide could destroy both tumor cells and tumor vasculature. The therapeutic effectiveness will be improved with safety.

If blood flow supplying tumor is fast, a balloon occlusion catheter can be used as shown on FIG. 4. During the injection of the gas. The balloon will be inflated to decrease or occlude the local blood flow temporary that benefits the gas accumulation within tumor vasculature. During the reperfusion, the balloon will be flatted to increase the blood flow into tumor vasculature that induces much more reperfusion injury. The blood supply fluctuation could be terminated when tumor vasculature is destroyed or vapor locked and no more blood supply into the tumor. Tumor apoptosis and necrosis will be achieved by the treatment. Due to the vapor lock, less inflammatory post the treatment is expected that make the patients have less fever, pain and discomfort.

### Example 4

### Blood Supply Fluctuation Therapy (BSFT) with Gases Mixture of CO₂ and Oxygen/Ozone for the Treatment of Splenomegaly

Preparation of gaseous mixture of gaseous mixture of CO₂ and Oxygen/Ozone: the preparation procedure will be same as that of gaseous mixture in Example 3. The concentration of oxygen will be present in an amount effective to decrease the absorbability of the gaseous mixture about 20-60%, preferably between 5% and 20%. The concentration of ozone will be present in an amount effective to prevention bacterial infection and kill virus in liver and portal vein, preferably 20-50 mg/L.

Selective catheterization and diagnostic Digital Subtraction Angiogrpahy (DSA) with Gaseous Mixture of CO₂ and Oxygen/Ozone: under monitoring of X-ray fluoroscope and using interventional technique of selective catheterization, a delivery catheter will be inserted into the splenic artery in patients with splenomegaly due to virus hepatitis cirrhosis and portal hypertension. The delivery catheter will be connected to the outlet of the third three-way stopcock of the delivery system, and the air within the catheter will be removed. Using the gaseous mixture as contrast medium, 30-50 ml gaseous mixture at rate of 10-15 ml/second will be injected by syringe into the splenic artery to perform a diagnostic DSA. The diagnostic DSA will be used to identify anatomy and blood flow of the spleen and time of the gaseous mixture staying in splenic tissue. All those information is necessary to doctor to select an optimal protocol of Blood Supply Fluctuation Therapy (BSFT). Then, the catheter will be advanced into the distal branches of the splenic artery.

Blood Supply Fluctuation Therapy (BSFT) with Gaseous Mixture of CO₂ and Oxygen/Ozone: The goals of the treatment here should be: decreasing the blood flow within splenic tissue to save more blood cells and platelets from destruction in spleen; to produce splenic apoptosis, not necrosis to limit post embolization syndrome; using ozone to kill bacterial and virus in portal vein and liver. Gaseous mixture of CO₂ and Oxygen/Ozone will be injected slowly and intermittently by the delivery syringe through catheter into splenic vasculature at the rate of 1-2 ml/s and with 3-5 minutes interval each 10 ml injection. The gas will displace the blood flow within the splenic tissue partially (40-80%). Because splenic vasculature is mature, “vapor lock” in splenic vasculature is not expected. With the decreasing the blood flow within splenic vasculature, less blood cells and platelets will pass through splenic vasculature and much more blood cells and platelets will be protected from destruction. Meanwhile the blood supply fluctuation therapy will generate apoptosis, but not necrosis by decreasing the blood flow. The patients receiving the injection of gaseous mixture will feel minor discomfort without major post embolization syndrome. Also the Ozone in the gaseous mixture will mix and oxidate blood in spleen and enter into portal vein and liver to kill the bacterial and virus in the portal vein and liver tissue. After the treatment the swell spleen will shrink, and decreased blood cells and platelets number will return to normal by preventing them from over-destruction in spleen.

The exemplary embodiment has been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.
1. Incue H, Miyazono N, Miyake S, Ueno K, Nishida H, Nakajo M: Staging hepatic tumors with CT and carbon dioxide. J Comput Assist Tomogr. November-December 1994;18(6):882-7.
2. Yang X, Manninen H, Soimakallio S: Carbon dioxide in vascular imaging and intervention. Acta Radiol. July 1995;36(4):330-7.
3. Hawkins I F Jr, Caridi J G: CO2 digital subtraction arteriography—advantages and current solutions for delivery and imaging. Cardiovasc Intervent Radiol. May-June 1995; 18(3):150-2.
4. Kerns S R, Hawkins I F Jr. Carbon dioxide digital subtraction angiography: expanding applications and technical evolution. AJR Am J Roentgenol. March 1995;164(3):735-41.
5. Arakawa A, Nishiharu T, Matsukawa T, Urata J, Hatanaka Y, Yamashita Y, Takahashi M, Kanemitsu K, Mihara M. Detection of hepatocellular carcinoma by intraarterially enhanced ultrasonography with CO2 microbubbles. Comparison with DSA, dynamic CT, and Lipiodol CT. Acta Radiol. May 1996;37(3 Pt 1):250-4.
6. Arakawa A, Nishiharu T, Matsukawa T, Urata J, Hatanaka Y, Yamashita Y, Takahashi M, Kanemitsu K, Mihara M. Detection of hepatocellular carcinoma by intraarterially enhanced ultrasonography with CO2 microbubbles. Comparison with DSA, dynamic CT, and Lipiodol CT. Acta Radiol. May 1996;37(3 Pt 1):250-4.
7. Rundback J H, Shah P M, Wong J, Babu S C, Rozenblit G, Poplausky M R: Livedo reticularis, rhabdomyolysis, massive intestinal infarction, and death after carbon dioxide arteriography. J Vasc Surg. August 1997;26(2):337-40.
8. Caridi J G, Hawkins I F Jr: CO2 digital subtraction angiography: potential complications and their prevention. J Vasc Interv Radiol. May-June 1997;8(3):383-91.
9. Back M R, Caridi J G, Hawkins I F Jr, Seeger J M: Angiography with carbon dioxide (CO2). Surg Clin North Am. August 1998;78(4):575-91.
10. Miyajima Y, Sakaguchi S, Tohara K: [Transcatheter arterial embolization of hepatocellular carcinoma using CO2-US] Nippon Rinsho. April 1998;56(4):990-3. [Article in Japanese]
11. Spinosa D J, Matsumoto A H, Angle J F, Hagspiel K D, Hooper T N: Transient mesenteric ischemia: a complication of carbon dioxide angiography. J Vasc Interv Radiol. July-August 1998;9(4):561-4.
12. Hawkins I F, Caridi J G: Carbon dioxide (CO2) digital subtraction angiography: 26-year experience at the University of Florida. Eur Radiol. 1998;8(3):391-402.
13. Chen R C, Wang C K, Chiang L C, Lo H Y, Duh S J, Chen W T, Tu H Y, Liao L Y, Wang C S, Chen P H: Intra-arterial carbon dioxide-enhanced ultrasonogram of hepatocellular carcinoma treated by transcatheter arterial embolization and percutaneous ethanol injection therapy. J Gastroenterol Hepatol. January 1998;13(1):41-6.
14. Dimakakos P B, Stefanopoulos T, Doufas A G, Papasava M, Gouliamos A, Mourikis D, Deligiorgi H: The cerebral effects of carbon dioxide during digital subtraction angiography in the aortic arch and its branches in rabbits. AJNR Am J Neuroradiol. February 1998; 19(2):261-6.
15. Culp W C, McCowan T C, Goertzen T C, Habbe T G: Carbon dioxide angiography: complications and pseudocomplications. J Vasc Interv Radiol. January 1999;10(1):100-1.
16. Yang L, Bettmann M A: Identification of the portal vein: wedge hepatic venography with CO2 or iodinated contrast medium. Acad Radiol. February 1999;6(2):89-93.
17. Song K, Cho D, Shinn K, Charlton E, Cho K: Gas dynamics in CO₂ angiography: in vitro evaluation in a circulatory system model. Invest Radiol. February 1999;34(2):151-5.
18. Kessel D O, Robertson I, Patel J 5th, Peters K, Taylor E J, Denton L J, Bambhra B: Carbon-dioxide-guided vascular interventions: technique and pitfalls. Cardiovasc Intervent Radiol. November-December 2002;25(6):476-83. Epub 2002 Jul. 23.
19. Krajina A, Lojik M, Chovanec V, Raupach J, Hulek P: Wedged hepatic venography for targeting the portal vein during TIPS: comparison of carbon dioxide and iodinated contrast agents. Cardiovasc Intervent Radiol. May-June 2002;25(3):171-5. Epub 2002 Feb. 25
20. Chen R C, Wang C K, Wang C S, Chen W T, Shih L S, Chiang L C, Chen P H: Depiction of vasculature in small hepatocellular carcinoma, and dysplastic nodules evaluated with carbon dioxide ultrasonography and angiography. Acta Radiol. January 2002;43(1):66-70.
21. Sugimoto C; Saito A; Kikuzato M; Hayashi N: Angioechographic evaluation of tumor thrombi and the effect of transcatheter arterial embolization in patients with hepatocellular carcinoma. J Gastroenterol 2002;37(5):363-8
22. Wacker F K, Maes R M, Jesberger J A, Nour S G, Duerk J L, Lewin J S: MR imaging-guided vascular procedures using CO2 as a contrast agent. AJR Am J Roentgenol. August 2003; 181(2):485-9.
23. Caridi J G, Hawkins I F Jr, Cho K, Sohn S Y, Langham M R Jr, Weichmann B N, Klioze S D: CO2 splenoportography: preliminary results. AJR Am J Roentgenol. May 2003; 180(5):1375-8
24. Cho K J, Cho D R: CO2 digital subtraction splenoportography with the “skinny” needle: experimental study in a swine model. Cardiovasc Intervent Radiol (United States), May-June 2003, 26(3) p273-6
25. Burke C T, Weeks S M, Mauro M A, Jaques P F: CO2 splenoportography for evaluating the splenic and portal veins before or after liver transplantation. J Vasc Interv Radiol. October 2004;15(10):1161-5.
26. Yamamoto K, Shiraki K, Nakanishi S, Fuke H, Hashimoto A, Shimizu A, Nakano T: Usefulness of digital subtraction imaging with Levovist in the diagnosis of hepatocellular carcinomas. Oncol Rep. January 2005;13(1):95-9.
27. Liss P, Eklof H, Hellberg O, Hagg A, Bostrom-Ardin A, Lofberg A M, Olsson U, Orndahl P, Nilsson H, Hansell P, Eriksson L G, Bergqvist D, Nyman R.: Renal effects of CO2 and iodinated contrast media in patients undergoing renovascular intervention: a prospective, randomized study. J Vasc Interv Radiol. January 2005;16(1):57-65.

1. A method of blood supply fluctuation therapy (BSFT), which comprises:
(i) providing a target tissue in a mammal that is intended to be treated;
(ii) selecting one or more delivery sites in the blood supply system to the target tissue;
(iii) releasing a predetermined amount of bio-absorbable agent in the selected one or more delivery sites;
(iv) inducing down-fluctuation of the blood supply to the target tissue;
(v) inducing up-fluctuation of the blood supply to the target tissue when the bio-absorbable agent within the target tissue is and/or has been bio-absorbed; and
(vi) optionally repeating steps (ii) to (v) at least once.

2. The method according to claim 1, in which the mammal is a human or an animal.

3. The method according to the claim 1, in which the tissue is from an organ selected from the group consisting of liver, spleen, pancreas, kidney, brain, spine, lungs, bone, heart, vessels, muscle, stomach, intestine, uterus, bladder, gland, skin.

4. The method according to the claim 1, in which the tissue is a normal or an abnormal tissue.

5. The method according to the claim 4, in which the abnormal tissue is tumor, vascular malformation, inflammatory tissue, hemangioma, or splenomegaly.

6. The method according to the claim 1, in which the bio-absorbable agent is in the form of solid, liquid, gas, or combination thereof.

7. The method according to the claim 1, in which the bio-absorbable agent is gas composition.

8. The method according to the claim 7, in which the gas composition comprises CO₂.

9. The method according to the claim 8, in which the CO₂ is medical grade CO₂.

10. The method according to the claim 7, in which the gas composition comprises O₂.

11. The method according to the claim 7, in which the gas composition comprises a mixture of CO₂ and O₂.

12. The method according to any one of claim 8, 10, or 11, in which the gas composition further comprises a gas selected from the group consisting of ozone, NO, ammonia, H₂S, N₂, inert gases such as helium, neon, xenon, argon, and krypton, radioactive isotope thereof, and mixture thereof.

13. The method according to the claim 7, in which the volume of the gas composition of from 5 ml to 10000 ml.

14. The method according to claim 1, which is conducted under the monitoring of Digital Subtraction Angiography (DSA), Computer Tomography(CT), MRI, or Ultrasound(US).

15. The method according to claim 1, in which the bio-absorbable agent is delivered by an intravascular route including intra-arterial route, intravenous route, intracapililary route, intracardial route, or combination thereof.

16. The method according to claim 1, in which the bio-absorbable agent is delivered by a catheter or dual lumen occlusive balloon catheter.

17. The method according to claim 1, in which the down-fluctuation is achieved by a vapor lock formation within the target tissue.

18. The method according to claim 1, in which the down-fluctuation of the blood supply induces ischemia, hypoxia, cell necrosis and/or apoptosis induced from ischemia in the target tissue.

19. The method according to claim 1, in which the up-fluctuation of the blood supply induces reperfusion injury, blood vessel damaging, induced cell necrosis and/or apoptosis, blood vessel dilation, or improved O₂ and nutrition availability in the target tissue.

20. An agent used for the Blood Supply Fluctuation Therapy of claim 1, which is bio-absorbable.

21. The agent according to claim 20, which is a gas composition.

22. The agent according to claim 21, in which the gas composition comprises CO₂.

23. The agent according to claim 22, in which the CO₂ is medical grade CO2.

24. The agent according to claim 21, in which the gas composition comprises O₂.

25. The agent according to claim 21, in which the gas composition comprises a mixture of CO₂ and O₂.

26. The agent according to any one of claim 22, 24, or 25, in which the gas composition further comprises a gas selected from the group consisting of ozone, NO, ammonia, H₂S, N₂, inert gases such as helium, neon, xenon, argon, and krypton, radioactive isotope thereof, and mixture thereof.

27. A device for storing and delivering gaseous BSFT agent, comprising a storing bag and a delivery system.

28. The device according to claim 27, in which the storing bag (3) has an inlet tubular member (2) and an outlet tubular member (4), one end of said inlet tubular member connects to one side of said bag, and another end of said inlet tubular member (2) connects to gas resource, one end of said outlet tubular member connects to another side of said storing bag and another end of outlet tubular member connects to said delivery system; and the delivery system comprises a first check valve (5), a “T” shape three-way connecting tubular member (8), a second check valve (7), connecting tubular member(9), a third check valve (10), three-way stopcock (11), two ends of the straight arm on the “T” shape three-way connecting tubular member connect to the first check valve (5) and the second check valve (7), the first check valve connects to outlet tubular member (4), the second check valve connects to connecting tubular member (9), the middle port of the “T” shape three-way connecting tubular member (8) connects to a pump (6); the downstream outlet of said connecting tubular member (9) connects to the third check valve (10), and the middle port of the stopcock (11) connects to the outlet of the third check valve (10).

29. The device according to claim 28, in which the outlet of the straight arm of the three-way stopcock (11) connects to angiographic catheter (13), guide wire (11) passes through the straight arm of the three-way stopcock (11) into angiographic catheter (13).

30. The device according to claim 29, in which the angiographic catheter (13) is conventional single lumen catheter or dual lumen occlusive balloon catheter.

31. The device according to claim 28, in which the first check valve (5) and the second check valve connect to two ends of the straight arm of the “T” shape connecting tubular member (8) respectively.

32. The device according to claim 28, in which the inlet of the tubular member (2) has a stopcock (1) or a valve (14).
